(19) United States
(12) Patent Application Publication
(10) Pub. No.: US 2023/0210946 A1
(43) Pub. Date: Jul. 6, 2023

(54) TREATMENT OF CANCER

(71) Applicant: Immutep S.A.S., SAINT-AUBIN (FR)

(72) Inventor: Frederic TRIEBEL, Orsay (FR)

(73) Assignee: Immutep S.A.S., SAINT-AUBIN (FR)

(21) Appl. No.: 17/928,048

(22) PCT Filed: Mar. 24, 2021
(86) PCT No.: PCT/EP2021/057588

§ 371 Date: Nov. 28, 2022

(30) Foreign Application Priority Data
May 28, 2020 (GB) 2008037.0
Sep. 23, 2020 (RU) 2020131384
Nov. 17, 2020 (GB) 2018062.6

(51) Int. Cl. A61K 38/17; A61K 31/337; A61P 35/00

(52) U.S. Cl. CPC A61K 38/1774; A61K 31/337; A61P 35/00

(57) ABSTRACT

This invention relates to the use of a LAG-3 protein or a derivative thereof and optionally a chemotherapy agent for the treatment of cancer in a subject. The subject may have one or more of a low monocyte count, a Luminal B breast cancer, an age of less than about 85 years, has been previously treated with a CDK4/6 inhibitor, and has not previously undergone treatment with a taxane chemotherapy. Suitably, the cancer is a breast cancer, such as a hormone receptor positive breast cancer.

### FIELD OF THE INVENTION

This invention relates to the use of a LAG-3 protein or derivative thereof for the treatment of cancer.

### BACKGROUND OF THE INVENTION

Over the past decade, PD-1 and CTLA-4 immune checkpoint inhibitors such as OPDIVO (nivolumab), KEYTRUDA (pembrolizumab) and YERVOY (ipilimumab) have become the standard of care therapies for many forms of cancer, however unfortunately, many patients still fail to respond to these modern medicines. In an effort to improve patient outcomes, significant work has been undertaken to investigate other immune checkpoints, such as LAG-3, TIM-3, VISTA, CD47, IDO and TIGIT. LAG-3 in particular has emerged as a promising checkpoint and a number of companies are developing new inhibitors that target this checkpoint. The aim of a LAG-3 inhibitor, as with the currently approved PD-1 and CTLA-4 inhibitors, is to block the down-regulation of the immune system i.e. taking the “brakes off” the body's immune processes. Significant work has also been undertaken to explore combinations of PD-1 and CTLA-4 immune checkpoint inhibitors with other approved or experimental therapies. Another type of active immunotherapy being investigated are antigen presenting cell (APC) activators. APC activators bind to antigen presenting cells such as dendritic cells, monocytes and macrophages via MHC II molecules. This activates the APCs causing them to become professional antigen presenting cells, thereby presenting antigen to the adaptive immune system. This leads to activation and proliferation of CD4+(helper) and CD8+(cytotoxic) T cells. Thus, the aim of APC activators is to “push the gas” on the body's immune system.

Eftilagimod alpha (IMP321 or efti), a soluble dimeric recombinant form of LAG-3, is a first-in-class APC activator under clinical development. By stimulating dendritic cells and other APCs through MHC class II molecules, IMP321 induces a powerful anti-cancer T cell response. IMP321 is described in WO 2009/044273, which also describes the use of IMP321 alone and in combination with a chemotherapy agent for the treatment of cancer. There remains a need in the art for improved cancer therapies and treatment regimens leading to better outcomes for patients. This is especially so for cancers where the prognosis for patients undertaking treatment with current medicines is poor.

### SUMMARY OF THE INVENTION

In one embodiment, the invention relates to a LAG-3 protein, or a derivative thereof that is able to bind to MHC class II molecules, for use in preventing, treating, or ameliorating a cancer in a subject with a low monocyte count.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a Luminal B breast cancer in a subject.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, for the prevention, treatment, or amelioration of a Luminal B breast cancer in a subject.

In a further embodiment, the invention provides a method of preventing, treating, or ameliorating a Luminal B breast cancer, the method comprising administering to a subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject with an age of less than about 85 years.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject that has been previously treated with a CDK4/6 inhibitor.

In another embodiment, the invention relates to the use of a LAG-3 protein, or a derivative thereof that is able to bind to MHC class II molecules, in the manufacture of a medicament for the prevention, treatment, or amelioration of a cancer in a subject that has been previously treated with a CDK4/6 inhibitor.

In yet another embodiment, the invention relates to the use of a LAG-3 protein, or a derivative thereof that is able to bind to MHC class II molecules, for the prevention, treatment, or amelioration of a cancer in a subject that has been previously treated with a CDK4/6 inhibitor.

In a further embodiment, the invention provides a method of preventing, treating, or ameliorating a cancer in a subject that has been previously treated with a CDK4/6 inhibitor, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules.

### BRIEF DESCRIPTION OF THE DRAWINGS

FIG. 1 illustrates the amino acid sequence of mature human LAG-3 protein. The four extracellular Ig superfamily domains are at amino acid residues: 1-149 (D1); 150-239 (D2); 240-330 (D3); and 331-412 (D4). The amino acid sequence of the extra-loop structure of the D1 domain of human LAG-3 protein is shown underlined in bold.

FIG. 2 shows progression free survival (PFS) estimates for patients receiving paclitaxel+IMP321 vs paclitaxel+placebo (for blinded independent investigator read (BICR) and investigator read).

FIG. 3 shows overall survival (OS) estimates for patients receiving paclitaxel+IMP321 vs paclitaxel+placebo (investigator read).

FIG. 4 shows PFS estimates for the low monocytes patient subgroup receiving paclitaxel+IMP321 vs paclitaxel+placebo (investigator read).

FIG. 5 shows OS estimates for the low monocytes patient subgroup receiving paclitaxel+IMP321 vs paclitaxel+placebo (investigator read).

FIG. 6 shows PFS estimates for the Luminal B patient subgroup receiving paclitaxel+IMP321 vs paclitaxel+placebo (investigator read).

FIG. 7 shows OS estimates for the Luminal B patient subgroup receiving paclitaxel+IMP321 vs paclitaxel+placebo (investigator read).

FIG. 8 shows PFS estimates for patients <65 years of age receiving paclitaxel+IMP321 vs paclitaxel+placebo (investigator read).

FIG. 9 shows OS estimates for patients <65 years of age receiving paclitaxel+IMP321 vs paclitaxel+placebo (investigator read).

FIG. 10 shows the number of CD4 and CD8 T cells in patients after receiving paclitaxel+IMP321 (darker shaded bars) vs paclitaxel+placebo (lighter shaded bars). *p<0.05 Wilcoxon.

FIG. 11 shows the correlation between OS (5_ 18 months versus >18 months) and the number of CD8 T cells in patients.

### DETAILED DESCRIPTION OF THE INVENTION

Methods of Treatment in Patient Subgroup with Low Starting Monocyte Count

In one embodiment, the invention relates to a LAG-3 protein, or a derivative thereof that is able to bind to MHC class II molecules, for use in preventing, treating, or ameliorating a cancer in a subject with a low monocyte count.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules.

Exemplary cancers that may be treated according to the invention include, but are not limited to, breast cancer, skin cancer, lung cancer (especially NSCLC), ovarian cancer, renal cancer, colon cancer, colorectal cancer, gastric cancer, esophageal cancer, pancreatic cancer, bladder cancer, urothelial cancer, liver cancer, melanoma (for example, metastatic malignant melanoma), prostate cancer (for example hormone refractory prostate adenocarcinoma), head and neck cancer (for example, head and neck squamous cell carcinoma), cervical cancer, thyroid cancer, glioblastoma, glioma, leukemia, lymphoma (for example, a B cell lymphoma), adrenal gland cancer, AIDS-associated cancer, alveolar soft part sarcoma, astrocytic tumor, bone cancer, brain and spinal cord cancer, metastatic brain tumor, carotid body tumor, chondrosarcoma, chordoma, chromophobe renal cell carcinoma, clear cell carcinoma, cutaneous benign fibrous histiocytoma, desmoplastic small round cell tumor, ependymoma, Ewing's tumor, extraskeletal myxoid chondrosarcoma, fibrogenesis imperfecta ossium, fibrous dysplasia of the bone, gallbladder or bile duct cancer, gestational trophoblastic disease, germ cell tumor, haematological malignancy, hepatocellular carcinoma, islet cell tumor, Kaposi's sarcoma, kidney cancer, lipoma/benign lipomatous tumor, liposarcoma/malignant lipomatous tumor, medulloblastoma, meningioma, Merkel cell carcinoma, multiple endocrine neoplasia, multiple myeloma, myelodysplasia syndrome, neuroblastoma, neuroendocrine tumor, papillary thyroid carcinoma, parathyroid tumor, pediatric cancer, peripheral nerve sheath tumor, phaeochromocytoma, pituitary tumor, prostate cancer, posterior uveal melanoma, rare hematologic disorder, renal metastatic cancer, rhabdoid tumor, rhabdomysarcoma, sarcoma, soft-tissue sarcoma, squamous cell cancer, stomach cancer, synovial sarcoma, testicular cancer, thymic carcinoma, thymoma, thyroid metastatic cancer, and uterine cancer.

In one embodiment, the cancer is a head and neck cancer. In another embodiment, the head and neck cancer is head and neck squamous cell carcinoma (HNSCC).

In one embodiment, the cancer is a lung cancer. In another embodiment, the lung cancer is non-small cell lung cancer (NSCLC).

In one embodiment, the cancer is a breast cancer. Suitably, the breast cancer is an adenocarcinoma of the breast.

According to embodiments of the invention, the cancer may have progressed to metastatic disease.

In another embodiment, the breast cancer is a hormone receptor-positive cancer (estrogen-receptor positive and/or progesterone-receptor positive), which may be HER2 positive or HER2 negative. In one embodiment, the hormone receptor-positive cancer is HER2 negative. The hormone receptor-positive cancer may be a hormone receptor-positive metastatic breast cancer. In an embodiment, the hormone receptor-positive cancer is hormone receptor-positive HER2 negative metastatic breast cancer.

In an embodiment, the hormone receptor-positive cancer is a Luminal B breast cancer. Luminal B breast cancer is hormone-receptor positive, and either HER2 positive or HER2 negative and has high levels of Ki-67. Luminal B cancers generally grow slightly faster than Luminal A cancers and their prognosis is slightly worse.

In one embodiment, the hormone receptor-positive cancer is HER2 negative and is the Luminal B sub-type. As with other embodiments of the invention, the hormone receptor-positive HER2 negative breast cancer with the Luminal B sub-type may have progressed to metastatic disease. Thus, in an embodiment, the hormone receptor-positive cancer is hormone receptor-positive HER2 negative metastatic breast cancer with the Luminal B sub-type.

In another embodiment, the hormone receptor-positive cancer is a Luminal A breast cancer. Luminal A breast cancer is hormone-receptor positive (estrogen-receptor positive and/or progesterone-receptor positive), HER2 negative, and has low levels of the protein Ki-67.

In yet another embodiment, the breast cancer is triple negative breast cancer (estrogen-receptor negative, progesterone-receptor negative and HER2 negative).

In a further embodiment, the breast cancer is HER2-enriched breast cancer. HER2-enriched breast cancer is hormone-receptor negative (estrogen-receptor negative and progesterone-receptor negative) and HER2 positive. HER2-enriched cancers tend to grow faster than luminal cancers and can have a worse prognosis.

In some embodiments, the LAG-3 protein or derivative thereof is administered parenterally (including by subcutaneous, intravenous, or intramuscular injection). In particular embodiments, the LAG-3 protein or derivative thereof is administered subcutaneously by injection.

According to certain embodiments of the invention, patients with a low starting monocyte count are selected for treatment. As defined herein, a “low monocyte count” is less than about 0.25×10⁹ cells/L of blood at baseline. “Baseline” means prior to commencement of treatment according to the invention.

Methods of Treatment in Luminal B Breast Cancer Subgroup

In one embodiment, the invention relates to a LAG-3 protein, or a derivative thereof that is able to bind to MHC class II molecules, for use in preventing, treating, or ameliorating a Luminal B breast cancer in a subject.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, for the prevention, treatment, or amelioration of a Luminal B breast cancer in a subject.

In a further embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules.

As explained herein, a Luminal B breast cancer is hormone-receptor positive, and either HER2 positive or HER2 negative and has high levels of Ki-67. Luminal B cancers generally grow slightly faster than Luminal A cancers and their prognosis is slightly worse.

In one embodiment, the Luminal B breast cancer is HER2 negative. In another embodiment, the Luminal B breast cancer is HER2 negative, and has progressed to metastatic disease. Thus, in the embodiments of the invention, the Luminal B breast cancer is HER2 negative metastatic breast cancer.

In another embodiment, the Luminal B breast cancer is HER2 positive. In another embodiment, the Luminal B breast cancer is HER2 positive, and has progressed to metastatic disease. Thus, in the embodiments of the invention, the Luminal B breast cancer is HER2 positive metastatic breast cancer.

In yet another embodiment, the subject has a Luminal B breast cancer and the subject also has a low monocyte count.

In an embodiment, the subject has a Luminal B breast cancer which is HER2 negative and the subject also has a low monocyte count. Alternatively, the subject has a Luminal B breast cancer which is HER2 positive and the subject also has a low monocyte count.

In one particular embodiment, the subject has metastatic Luminal B breast cancer which is HER2 negative and the subject also has a low monocyte count.

Methods of Treatment in Age Based Patient Subgroup

In one embodiment, the invention relates to a LAG-3 protein, or a derivative thereof that is able to bind to MHC class II molecules, for use in preventing, treating, or ameliorating a cancer in a subject with an age of less than about 85 years.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules.

Suitably, the subject has an age of less than about 85 years, less than about 80 years, less than about 75 years, less than about 70 years, less than about 65 years, less than about 60 years, less than about 55 years, less than about 50 years, less than about 45 years, or less than about 40 years.

In one embodiment, the subject has an age between about 18 years and about 85 years. In another embodiment, the subject has an age between about 18 years and about 80 years. In yet another embodiment, the subject has an age between about 18 years and about 75 years. In a further embodiment, the subject has an age between about 18 years and about 70 years. In yet a further embodiment, the subject has an age between about 18 years and about 65 years. In an embodiment, the subject has an age between about 18 years and about 60 years. In another embodiment, the subject has an age between about 18 years and about 55 years. In yet another embodiment, the subject has an age between about 18 years and about 50 years. In a further embodiment, the subject has an age between about 18 years and about 45 years. In yet a further embodiment, the subject has an age between about 18 years and about 40 years.

In another embodiment, the subject is pre-menopausal.

Suitably, the subject has an age of less than about 85 years, less than about 84 years, less than about 83 years, less than about 82 years, less than about 81 years, less than about 80 years, less than about 79 years, less than about 78 years, less than about 77 years, less than about 76 years, less than about 75 years, less than about 74 years, less than about 73 years, less than about 72 years, less than about 71 years, less than about 70 years, less than about 69 years, less than about 68 years, less than about 67 years, less than about 66 years, less than about 65 years, less than about 64 years, less than about 63 years, less than about 62 years, less than about 61 years, less than about 60 years, less than about 59 years, less than about 58 years, less than about 57 years, less than about 56 years, or less than about 55 years, less than about 54 years, less than about 53 years, less than about 52 years, less than about 51 years, less than about 50 years, less than about 49 years, less than about 48 years, less than about 47 years, less than about 46 years, less than about 45 years, less than about 44 years, less than about 43 years, less than about 42 years, less than about 41 years, or less than about 40 years.

In each case, the patient may optionally be older than about 18 years and less than the age recited herein.

In one particular embodiment, the subject has an age of less than about 65 years.

In another particular embodiment, the subject has an age of less than about 53 years.

Exemplary cancers that may be treated according to this embodiment of the invention include, but are not limited to, those that are described hereinabove.

Methods of Treatment in Patient Subgroup Previously Treated with a CDK4/6 Inhibitor

In one embodiment, the invention relates to a LAG-3 protein, or a derivative thereof that is able to bind to MHC class II molecules, for use in preventing, treating, or ameliorating a cancer in a subject that has been previously treated with a CDK4/6 inhibitor.

In another embodiment, the invention relates to the use of a LAG-3 protein, or a derivative thereof that is able to bind to MHC class II molecules, in the manufacture of a medicament for the prevention, treatment, or amelioration of a cancer in a subject that has been previously treated with a CDK4/6 inhibitor.

In yet another embodiment, the invention relates to the use of a LAG-3 protein, or a derivative thereof that is able to bind to MHC class II molecules, for the prevention, treatment, or amelioration of a cancer in a subject that has been previously treated with a CDK4/6 inhibitor.

In a further embodiment, the invention provides a method of preventing, treating, or ameliorating a cancer in a subject that has been previously treated with a CDK4/6 inhibitor, the method comprising administering to the subject in need of such prevention, treatment, or amelioration a LAG-3 protein, or a derivative thereof that is able to bind to MHC class II molecules.

CDK4/6 inhibitors are a new class of treatments for cancer, especially hormone receptor positive HER2 negative metastatic breast cancer, that target cyclin-dependent kinase 4 and 6. Exemplary CDK4/6 inhibitors include, but are not limited to, palbociclib, ribociclib and abemaciclib.

Suitably, in an embodiment of the invention, the subject has previously undertaken therapy with a CDK4/6 inhibitor, but their disease has continued to progress and they require an alternative treatment option.

Exemplary cancers that may be treated according to this embodiment of the invention include, but are not limited to, those that are described hereinabove.

In one embodiment, the cancer is a breast cancer. In another embodiment, the breast cancer is hormone receptor positive HER2 negative (HR+/HER2−) breast cancer.

In yet another embodiment, the hormone receptor positive HER2 negative breast cancer is hormone receptor positive HER2 negative metastatic breast cancer.

Methods of Treatment in Patient Subgroup Not Previously Treated with Taxane Therapy

In one embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules.

Suitably, in an embodiment of the invention, the subject has not previously undergone treatment with a taxane chemotherapy. Taxane chemotherapy agents feature a taxadiene structure, and act by binding to tubulin, thus stabilizing the microtubule polymer and protecting it from disassembly. This in turn blocks the progression of mitosis, triggering apoptosis (cell death). Taxane chemotherapies are effective in a wide variety of cancers including breast, ovarian, lung, pancreatic, prostate, and head and neck cancers.

Exemplary taxane chemotherapies include, but are not limited to, paclitaxel, docetaxel, cabazitaxel, larotaxel, milataxel, ortataxel, taxoprexin, opaxio, tesetaxel, and BMS-184476.

Exemplary cancers that may be treated according to this embodiment of the invention include, but are not limited to, those that are described hereinabove.

Methods of Treatment in One or More Subgroups

In one embodiment, the subject has one or more of a low monocyte count, a Luminal B breast cancer, an age of less than about 85 years, has been previously treated with a CDK4/6 inhibitor, and has not previously undergone treatment with a taxane chemotherapy.

Suitably, the subject has a low monocyte count and a Luminal B breast cancer.

Suitably, the subject has a low monocyte count and an age of less than about 85 years.

Suitably, the subject has a low monocyte count and has been previously treated with a CDK4/6 inhibitor.

Suitably, the subject has a low monocyte count and has not previously undergone treatment with a taxane chemotherapy.

Suitably, the subject has a Luminal B breast cancer and an age of less than about 85 years.

Suitably, the subject has a Luminal B breast cancer and has been previously treated with a CDK4/6 inhibitor.

Suitably, the subject has a Luminal B breast cancer and has not previously undergone treatment with a taxane chemotherapy.

Suitably, the subject has an age of less than about 85 years and has been previously treated with a CDK4/6 inhibitor.

Suitably, the subject has an age of less than about 85 years and has not previously undergone treatment with a taxane chemotherapy.

Suitably, the subject has been previously treated with a CDK4/6 inhibitor and has not previously undergone treatment with a taxane chemotherapy.

Suitably, the subject has a low monocyte count, a Luminal B breast cancer, and an age of less than about 85 years.

Suitably, the subject has a low monocyte count, a Luminal B breast cancer, and has been previously treated with a CDK4/6 inhibitor.

Suitably, the subject has a low monocyte count, a Luminal B breast cancer, and has not previously undergone treatment with a taxane chemotherapy.

Suitably, the subject has a low monocyte count, an age of less than about 85 years, and has been previously treated with a CDK4/6 inhibitor.

Suitably, the subject has a low monocyte count, an age of less than about 85 years, and has not previously undergone treatment with a taxane chemotherapy.

Suitably, the subject has a low monocyte count, has been previously treated with a CDK4/6 inhibitor, and has not previously undergone treatment with a taxane chemotherapy.

Suitably, the subject has a Luminal B breast cancer, an age of less than about 85 years, and has been previously treated with a CDK4/6 inhibitor.

Suitably, the subject has a Luminal B breast cancer, an age of less than about 85 years, and has not previously undergone treatment with a taxane chemotherapy.

Suitably, the subject has an age of less than about 85 years, has been previously treated with a CDK4/6 inhibitor, and has not previously undergone treatment with a taxane chemotherapy.

Suitably, the subject has a low monocyte count, a Luminal B breast cancer, an age of less than about 85 years, and has been previously treated with a CDK4/6 inhibitor.

Suitably, the subject has a low monocyte count, a Luminal B breast cancer, an age of less than about 85 years, and has not previously undergone treatment with a taxane chemotherapy.

Suitably, the subject has a low monocyte count, a Luminal B breast cancer, has been previously treated with a CDK4/6 inhibitor, and has not previously undergone treatment with a taxane chemotherapy.

Suitably, the subject has a low monocyte count, an age of less than about 85 years, has been previously treated with a CDK4/6 inhibitor, and has not previously undergone treatment with a taxane chemotherapy.

Suitably, the subject has a low monocyte count, a Luminal B breast cancer, an age of less than about 85 years, has been previously treated with a CDK4/6 inhibitor, and has not previously undergone treatment with a taxane chemotherapy.

In one particular embodiment, the subject has one or more of a low monocyte count, an age of less than about 85 years, and has not previously undergone treatment with a taxane chemotherapy.

In another particular embodiment, the subject has been previously treated with a CDK4/6 inhibitor and has one or more of a low monocyte count, an age of less than about 85 years, and has not previously undergone treatment with a taxane chemotherapy.

LAG-3 Protein and Derivatives

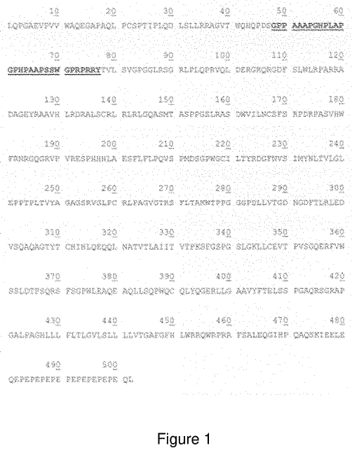

According to embodiments of the invention, the LAG-3 protein may be an isolated natural or recombinant LAG-3 protein. The LAG-3 protein may comprise an amino acid sequence of LAG-3 protein from any suitable species, such as a primate or murine LAG-3 protein, but preferably a human LAG-3 protein. The amino acid sequence of human and murine LAG-3 protein is provided in FIG. 1 of Huard et al (Proc. Natl. Acad. Sci. USA, 11: 5744-5749, 1997). The sequence of human LAG-3 protein is repeated in FIG. 1 herein (SEQ ID NO: 1). The amino acid sequences of the four extracellular Ig superfamily domains (D1, D2, D3, and D4) of human LAG-3 are also identified in FIG. 1 of Huard et al., at amino acid residues: 1-149 (D1); 150-239 (D2); 240-330 (D3); and 331-412 (D4).

Derivatives of LAG-3 protein include soluble fragments, variants, or mutants of LAG-3 protein that are able to bind to MHC class II molecules. Several derivatives of LAG-3 protein are known that are able to bind to MHC class II molecules. Many examples of such derivatives are described in Huard et al (Proc. Natl. Acad. Sci. USA, 11: 5744-5749, 1997). This document describes characterization of the MHC class II binding site on LAG-3 protein. Methods for making mutants of LAG-3 are described, as well as a quantitative cellular adhesion assay for determining the ability of LAG-3 mutants to bind to class II-positive Daudi cells. Binding of several different mutants of LAG-3 to MHC class II molecules was determined. Some mutations were able to reduce class II binding, while other mutations increased the affinity of LAG-3 for class II molecules. Many of the residues essential for binding of LAG-3 to MHC class II proteins are clustered at the base of a large 30 amino acid extra-loop structure in the LAG-3 D1 domain. The amino acid sequence of the extra-loop structure of the D1 domain of human LAG-3 protein is GPPAAAPGHPLAPGPHPAAPSSWGPRPRRY (SEQ ID NO:2). The amino acid sequence of the extra-loop structure of the D1 domain of human LAG-3 protein is shown underlined in bold in FIG. 1.

In an embodiment of the invention, the derivative of LAG-3 protein comprises the 30 amino acid extra-loop sequence of the human LAG-3 D1 domain, or a variant of such sequence with one or more conservative amino acid substitutions. The variant may comprise an amino acid sequence that has at least 70%, 80%, 90%, or 95% amino acid identity with the 30 amino acid extra-loop sequence of the human LAG-3 D1 domain.

The derivative of LAG-3 protein may comprise an amino acid sequence of domain D1, domain D1 and optionally D2, or domains D1 and D2, of LAG-3 protein, preferably human LAG-3 protein.

The derivative of LAG-3 protein may comprise an amino acid sequence that has at least 70%, 80%, 90%, or 95% amino acid identity with domain D1, domain D1 and optionally D2, or domains D1 and D2, of LAG-3 protein, preferably human LAG-3 protein.

The derivative of LAG-3 protein may comprise an amino acid sequence of domains D1, D2, and D3, domains D1, D2, D3 and optionally D4, or domains D1, D2, D3 and D4, of LAG-3 protein, preferably human LAG-3 protein.

The derivative of LAG-3 protein may comprise an amino acid sequence that has at least 70%, 80%, 90%, or 95% amino acid identity with domains D1, D2 and D3, domains D1, D2, D3 and optionally D4, or with domains D1, D2, D3 and D4, of LAG-3 protein, preferably human LAG-3.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

The derivative of LAG-3 protein may be fused to Immunoglobulin Fc amino acid sequence, preferably human IgG1 Fc amino acid sequence, optionally by a linker amino acid sequence.

The ability of a derivative of LAG-3 protein to bind to MHC class II molecules may be determined using a quantitative cellular adhesion assay as described in Huard et al (Proc. Natl. Acad. Sci. USA, 11: 5744-5749, 1997). The affinity of a derivative of LAG-3 protein for MHC class II molecules may be at least 20%, 30%, 40%, 50%, 60%, 70%, 80%, 90%, or 100% of the affinity of human LAG-3 protein for MHC class II molecules.

Preferably, the affinity of a derivative of LAG-3 protein for MHC class II molecules is at least 50%, 60%, 70%, 80%, 90%, 95%, 99%, or 100% of the affinity of human LAG-3 protein for MHC class II molecules.

Examples of suitable derivatives of LAG-3 protein that are able to bind to MHC class II molecules include derivatives comprising:
amino acid residues 23 to 448 of the human LAG-3 sequence;
amino acid sequence of domains D1 and D2 of LAG-3;
amino acid sequence of domains D1 and D2 of LAG-3 with an amino acid substitution at one or more of the following positions: position 30 where ASP is substituted with ALA; position 56 where HIS is substituted with ALA; position 73 where ARG is substituted with GLU; position 75 where ARG is substituted with ALA or GLU; position 76 where ARG is substituted with GLU; or position 103 where ARG is substituted with ALA; and
a recombinant soluble human LAG-31g fusion protein (IMP321)—a 160-kDa dimer produced in Chinese hamster ovary cells transfected with a plasmid encoding for the extracellular domain of hLAG-3 fused to the human IgG1 Fc. The sequence of IMP321 is given in SEQ ID NO: 17 of US 2011/0008331.

In an embodiment, the subject is a mammal, preferably a human.

According to the invention, the LAG-3 protein or derivative thereof is administered in a therapeutically effective amount. A “therapeutically effective amount” refers to an amount of the active ingredient sufficient to have a therapeutic effect upon administration. Effective amounts of the active ingredient will vary, for example, with the particular disease or diseases being treated, the severity of the disease, the duration of the treatment, and characteristics of the patient (e.g. sex, age, height and weight).

In an embodiment, the LAG-3 protein or derivative thereof is administered at a dose which is a molar equivalent of about 0.1 mg to about 60 mg, about 6 mg to about 60 mg, about 10 mg to about 50 mg, about 20 mg to about 40 mg, about 25 mg to about 35 mg, or about 30 mg of the LAG-3 derivative LAG-31g fusion protein IMP321.

In other embodiments, the LAG-3 protein or derivative thereof is administered at a dose which is a molar equivalent of about 0.25 mg to about 30 mg, about 1 mg to about 30 mg, or about 6 mg to about 30 mg of the LAG-3 derivative LAG-31g fusion protein IMP321.

In another embodiment, the LAG-3 protein or derivative thereof is administered at a dose which is a molar equivalent of about 25 mg, about 26 mg, about 27 mg, about 28 mg, about 29 mg, about 30 mg, about 31 mg, about 32 mg, about 33 mg, about 34 mg, or about 35 mg of the LAG-3 derivative LAG-31g fusion protein IMP321.

Suitably, the LAG-3 protein or derivative thereof is administered at a dose which is a molar equivalent of about 30 mg of the LAG-3 derivative LAG-31g fusion protein IMP321.

In yet another embodiment, the LAG-3 protein or derivative thereof is administered at a dose which is a molar equivalent from about 25 mg to about 60 mg, such as about 25 mg, about 30 mg, about 35 mg, about 40 mg, about 45 mg, about 50 mg, about 55 mg, or about 60 mg, of the LAG-3 derivative LAG-31g fusion protein IMP321.

In one embodiment, the LAG-3 protein or derivative thereof is IMP321 and is administered at a dose of about 0.1 mg to about 60 mg, about 6 mg to about 60 mg, about 10 mg to about 50 mg, about 20 mg to about 40 mg, about 25 mg to about 35 mg, or about 30 mg.

In another embodiment, the IMP321 is administered at a dose of about 25 mg, about 26 mg, about 27 mg, about 28 mg, about 29 mg, about 30 mg, about 31 mg, about 32 mg, about 33 mg, about 34 mg, or about 35 mg.

Suitably, IMP321 is administered at a dose of about 30 mg.

In other embodiments, IMP321 is administered at a dose from about 25 mg to about 60 mg, such as about 25 mg, about 30 mg, about 35 mg, about 40 mg, about 45 mg, about 50 mg, about 55 mg, or about 60 mg.

Doses of 6-30 mg per subcutaneous (s.c.) injection of IMP321 have been shown, thus far, to be safe and provide an acceptable systemic exposure based on the results of pharmacokinetics data obtained in metastatic renal cell cancer patients. A blood concentration of IMP321 superior to 1 ng/ml for at least 24 hours after s.c. injection is obtained in patients injected with IMP321 doses of more than 6 mg. No dose limiting toxicity has been observed to date.

In an embodiment, the LAG-3 protein or derivative thereof is administered about once every week to the subject. In another embodiment, the LAG-3 protein or derivative thereof is administered about once every two weeks to the subject. In yet another embodiment, the LAG-3 protein or derivative thereof is administered about once every three weeks to the subject. In a further embodiment, the LAG-3 protein or derivative thereof is administered about once every four weeks to the subject. In yet a further embodiment, the LAG-3 protein or derivative thereof is administered about once every month to a subject. As will be appreciated by those of skill in the art, the precise treatment regimen will vary and be adapted according to the particular cancer being treated and characteristics of the patient.

In one embodiment, the LAG-3 protein or derivative thereof is present as the sole active ingredient. In another embodiment, the LAG-3 protein or derivative thereof is present in the absence of any additional antigen added to the pharmaceutical composition or medicament.

Combination Treatment with Chemotherapy in One or More Subgroups

In an embodiment, the LAG-3 protein or derivative thereof is administered in combination with a chemotherapy agent.

Suitable chemotherapy agents include, but are not limited to, alkylating agents, plant alkaloids, antitumor antibiotics, antimetabolites, topoisomerase inhibitors, and miscellaneous antineoplastics.

Suitably, the chemotherapy agent is an alkylating agent. Exemplary alkylating agents include mustard gas derivatives such mechlorethamine, cyclophosphamide, chlorambucil, melphalan, and ifosfamide; ethylenimines such as thiotepa and hexamethylmelamine; alkylsulfonates such as busulfan; hydrazines and triazines such as altretamine, procarbazine, dacarbazine and temozolomide; nitrosureas such as carmustine, lomustine and streptozocin; and metal salts such as carboplatin, cisplatin, and oxaliplatin.

Suitably, the chemotherapy agent is a plant alkaloid. Exemplary plant alkaloids include vinca alkaloids such as vincristine, vinblastine and vinorelbine; taxanes such as paclitaxel, docetaxel, cabazitaxel, larotaxel, milataxel, ortataxel, taxoprexin, opaxio, tesetaxel, and BMS-184476; podophyllotoxins such as etoposide and tenisopide; and camptothecan analogs such as irinotecan and topotecan.

Suitably, the chemotherapy agent is an antitumor antibiotic. Exemplary antitumor antibiotics include anthracyclines such as doxorubicin, daunorubicin, epirubicin, mitoxantrone, and idarubicin; chromomycins such as dactinomycin and plicamycin; and miscellaneous antitumor antibiotics such as mitomycin and bleomycin.

Suitably, the chemotherapy agent is an antimetabolite. Exemplary antimetabolites include folic acid antagonists such as methotrexate; pyrimidine antagonists such as 5-fluorouracil, foxuridine, cytarabine, capecitabine and gemcitabine; purine antagonists such as 6-mercaptopurine and 6-thioguanine; and adenosine deaminase inhibitors such as cladribine, fludarabine, nelarabine and pentostatin.

Suitably, the chemotherapy agent is a topoisomerase inhibitor. Exemplary topoisomerase inhibitors include topoisomerase I inhibitors such as irinotecan and topotecan; and topoisomerase II inhibitors such as amsacrine, etoposide, etoposide phosphate and teniposide.

Suitably, the chemotherapy agent is a miscellaneous antineoplastic.

Exemplary miscellaneous antineoplastics include ribonucleotide reductase inhibitors such as hydroxyurea; adrenocortical steroid inhibitors such as mitotane; enzymes such as asparaginase and pegaspargase; antimicrotubule agents such as estramustine; and retinoids such bexarotene, isotretinoin and tretinoin.

In one particular embodiment, the chemotherapy agent is a taxane. In an embodiment, the taxane is paclitaxel, docetaxel, cabazitaxel, larotaxel, milataxel, ortataxel, taxoprexin, opaxio, tesetaxel, or BMS-184476. In another embodiment, the taxane is paclitaxel.

The chemotherapy agent is administered in a therapeutically effective amount. A therapeutically effective amount refers to an amount of the chemotherapy agent sufficient to have a therapeutic effect upon administration. Effective amounts of the chemotherapy agent will vary with the chemotherapy agent selected, the particular disease or diseases being treated, the severity of the disease, the duration of the treatment, and characteristics of the patient (e.g. sex, age, height and weight).

In some embodiments, the chemotherapy agent is administered parenterally (including by subcutaneous, intravenous, or intramuscular injection) or orally. Suitably, the chemotherapy is administered intravenously.

In an embodiment, the LAG-3 protein or derivative thereof is administered before, with or after administration of the chemotherapy agent. In another embodiment, the LAG-3 protein or derivative thereof is administered after administration of the chemotherapy agent.

In one embodiment, the LAG-3 protein or derivative thereof and the chemotherapy agent are packaged separately. That is, in this embodiment, the LAG-3 protein or derivative thereof and the chemotherapy agent are separate unit dosage forms, which would typically (but not necessarily) be sourced from different suppliers, and then used in the methods of the invention.

In another embodiment, the LAG-3 protein or derivative thereof and the chemotherapy agent are in the form of a combined preparation.

The components of the “combined preparation” may be present: (i) in one combined unit dosage form known as a fixed dose combination (FDC), or (ii) as a first unit dosage form of component (a) and a separate, second unit dosage form of component (b) packaged together known as a kit-of-parts. The ratio of the total amounts of the combination component (a) to the combination component (b) to be administered in the combined preparation can be varied, for example, in order to cope with the needs of a patient sub-population to be treated, or the needs of the patient, which can be due, for example, to the particular disease, age, sex, or body weight of the patient.

That is, the combined preparation according to the invention may take the form of a pharmaceutical composition comprising the LAG-3 protein or derivative thereof and the chemotherapy agent or, alternatively, as a kit-of-parts comprising the LAG-3 protein or derivative thereof and the chemotherapy agent as separate components, but packaged together.

The kit-of-parts may comprise a plurality of doses of the LAG-3 protein or derivative thereof and/or a plurality of doses of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject with a low monocyte count.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, and a chemotherapy agent, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject with a low monocyte count, wherein the LAG-3 protein, or a derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count, wherein the LAG-3 protein or derivative thereof is to be administered before, with or after administration of a chemotherapy agent.

In an embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a Luminal B breast cancer in a subject.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, and a chemotherapy agent, for the prevention, treatment, or amelioration of a Luminal B breast cancer in a subject.

In a further embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a Luminal B breast cancer in a subject, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject, wherein the LAG-3 protein or derivative thereof is to be administered before, with or after administration of a chemotherapy agent.

In an embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject with an age of less than about 85 years.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, and a chemotherapy agent, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject with an age of less than about 85 years, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years, wherein the LAG-3 protein or derivative thereof is to be administered before, with or after administration of a chemotherapy agent.

In an embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject previously treated with a CDK4/6 inhibitor.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject previously treated with a CDK4/6 inhibitor.

In yet another embodiment, the invention relates to the use of a LAG-3 protein, or a derivative thereof that is able to bind to MHC class II molecules, and a chemotherapy agent, for the prevention, treatment, or amelioration of a cancer in a subject previously treated with a CDK4/6 inhibitor.

In a further embodiment, the invention provides a method of preventing, treating, or ameliorating a cancer in a subject previously treated with a CDK4/6 inhibitor, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject previously treated with a CDK4/6 inhibitor, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject previously treated with a CDK4/6 inhibitor, wherein the LAG-3 protein or derivative thereof is to be administered before, with or after administration of a chemotherapy agent.

In an embodiment, the invention provides a method of preventing, treating, or ameliorating a cancer in a subject previously treated with a CDK4/6 inhibitor, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, and a chemotherapy agent, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy, wherein the LAG-3 protein or derivative thereof is to be administered before, with or after administration of a chemotherapy agent.

In an embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject, wherein the subject has one or more of a low monocyte count, a Luminal B breast cancer, an age of less than about 85 years, has been previously treated with a CDK4/6 inhibitor, and has not previously undergone treatment with a taxane chemotherapy.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject, wherein the subject has one or more of a low monocyte count, a Luminal B breast cancer, an age of less than about 85 years, has been previously treated with a CDK4/6 inhibitor, and has not previously undergone treatment with a taxane chemotherapy.

In yet another embodiment, the invention relates to the use of a LAG-3 protein, or a derivative thereof that is able to bind to MHC class II molecules, and a chemotherapy agent, for the prevention, treatment, or amelioration of a cancer in a subject, wherein the subject has one or more of a low monocyte count, a Luminal B breast cancer, an age of less than about 85 years, has been previously treated with a CDK4/6 inhibitor, and has not previously undergone treatment with a taxane chemotherapy.

In a further embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the subject has one or more of a low monocyte count, a Luminal B breast cancer, an age of less than about 85 years, has been previously treated with a CDK4/6 inhibitor, and has not previously undergone treatment with a taxane chemotherapy.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered before, with or after administration of a chemotherapy agent, and wherein the subject has one or more of a low monocyte count, a Luminal B breast cancer, an age of less than about 85 years, has been previously treated with a CDK4/6 inhibitor, and has not previously undergone treatment with a taxane chemotherapy.

In another embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered before, with or after administration of a chemotherapy agent, and wherein the subject has one or more of a low monocyte count, a Luminal B breast cancer, an age of less than about 85 years, has been previously treated with a CDK4/6 inhibitor, and has not previously undergone treatment with a taxane chemotherapy.

In an embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wheren the LAG-3 protein or derivative thereof is administered before, with or after administration of the chemotherapy agent, and wherein the subject has one or more of a low monocyte count, a Luminal B breast cancer, an age of less than about 85 years, has been previously treated with a CDK4/6 inhibitor, and has not previously undergone treatment with a taxane chemotherapy.

Suitably, the subject has a low monocyte count and a Luminal B breast cancer.

Suitably, the subject has a low monocyte count and an age of less than about 85 years.

Suitably, the subject has a low monocyte count and has been previously treated with a CDK4/6 inhibitor.

Suitably, the subject has a low monocyte count and has not previously undergone treatment with a taxane chemotherapy.

Suitably, the subject has a Luminal B breast cancer and an age of less than about 85 years.

Suitably, the subject has a Luminal B breast cancer and has been previously treated with a CDK4/6 inhibitor.

Suitably, the subject has a Luminal B breast cancer and has not previously undergone treatment with a taxane chemotherapy.

Suitably, the subject has an age of less than about 85 years and has been previously treated with a CDK4/6 inhibitor.

Suitably, the subject has an age of less than about 85 years and has not previously undergone treatment with a taxane chemotherapy.

Suitably, the subject has been previously treated with a CDK4/6 inhibitor and has not previously undergone treatment with a taxane chemotherapy.

Suitably, the subject has a low monocyte count, a Luminal B breast cancer, and an age of less than about 85 years.

Suitably, the subject has a low monocyte count, a Luminal B breast cancer, and has been previously treated with a CDK4/6 inhibitor.

Suitably, the subject has a low monocyte count, a Luminal B breast cancer, and has not previously undergone treatment with a taxane chemotherapy.

Suitably, the subject has a low monocyte count, an age of less than about 85 years, and has been previously treated with a CDK4/6 inhibitor.

Suitably, the subject has a low monocyte count, an age of less than about 85 years, and has not previously undergone treatment with a taxane chemotherapy.

Suitably, the subject has a low monocyte count, has been previously treated with a CDK4/6 inhibitor, and has not previously undergone treatment with a taxane chemotherapy.

Suitably, the subject has a Luminal B breast cancer, an age of less than about 85 years, and has been previously treated with a CDK4/6 inhibitor.

Suitably, the subject has a Luminal B breast cancer, an age of less than about 85 years, and has not previously undergone treatment with a taxane chemotherapy.

Suitably, the subject has an age of less than about 85 years, has been previously treated with a CDK4/6 inhibitor, and has not previously undergone treatment with a taxane chemotherapy.

Suitably, the subject has a low monocyte count, a Luminal B breast cancer, an age of less than about 85 years, and has been previously treated with a CDK4/6 inhibitor.

Suitably, the subject has a low monocyte count, a Luminal B breast cancer, an age of less than about 85 years, and has not previously undergone treatment with a taxane chemotherapy.

Suitably, the subject has a low monocyte count, a Luminal B breast cancer, has been previously treated with a CDK4/6 inhibitor, and has not previously undergone treatment with a taxane chemotherapy.

Suitably, the subject has a low monocyte count, an age of less than about 85 years, has been previously treated with a CDK4/6 inhibitor, and has not previously undergone treatment with a taxane chemotherapy.

Suitably, the subject has a low monocyte count, a Luminal B breast cancer, an age of less than about 85 years, has been previously treated with a CDK4/6 inhibitor, and has not previously undergone treatment with a taxane chemotherapy.

In one particular embodiment, the subject has one or more of a low monocyte count, an age of less than about 85 years, and has not previously undergone treatment with a taxane chemotherapy.

In another particular embodiment, the subject has been previously treated with a CDK4/6 inhibitor and has one or more of a low monocyte count, an age of less than about 85 years, and has not previously undergone treatment with a taxane chemotherapy.

In one embodiment, the invention relates to a LAG-3 protein, or a derivative thereof that is able to bind to MHC class II molecules, for use in preventing, treating, or ameliorating a hormone receptor-positive breast cancer in a subject with an age of less than about 65 years, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for preventing, treating, or ameliorating a hormone receptor-positive breast cancer in a subject with an age of less than about 65 years, wherein the LAG-3 protein or derivative thereof is to be administered before, with or after administration of a chemotherapy agent.

In yet another embodiment, the invention relates to the use of a LAG-3 protein, or a derivative thereof that is able to bind to MHC class II molecules, for the prevention, treatment, or amelioration of a hormone receptor-positive breast cancer in a subject with an age of less than about 65 years, wherein the LAG-3 protein or derivative thereof is to be administered before, with or after administration of a chemotherapy agent.

In a further embodiment, the invention provides a method of preventing, treating, or ameliorating a hormone receptor-positive breast cancer in a subject with an age of less than about 65 years, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the LAG-3 protein or derivative thereof is administered to the subject after administration of the chemotherapy agent and within about 12 to about 96 hours, about 12 to about 48 hours, or about 24 hours, of administration of the chemotherapy agent.

According to another embodiment of the invention, combination treatment with chemo-immunotherapy comprises 6 cycles of 4 weeks. Patients receive weekly paclitaxel at Days 1, 8 and 15 with adjunctive treatment with the LAG-3 protein or derivative thereof on Days 2 and 16 of each 4-week cycle. After completion of the 6-cycle chemo-immunotherapy phase, responding or stable patients receive LAG-3 protein or derivative thereof every 4 weeks during a maintenance phase for an additional period of up to 12 injections (48 weeks).

In another embodiment, the chemo-immunotherapy combination treatment comprises 7 cycles of 4 weeks, or 8 cycles of 4 weeks, or 9 cycles of 4 weeks, or 10 cycles of 4 weeks, or 11 cycles of 4 weeks, or 12 cycles of 4 weeks (extended combination treatment).

In other embodiments, after the chemo-immunotherapy phase, responding or stable patients receive LAG-3 protein or derivative thereof about every week, or about every 2 weeks, or about every 3 weeks, during a maintenance phase for an additional period of up to about 48 weeks.

In another embodiment, the invention relates to the use of a LAG-3 protein or derivative thereof as a maintenance therapy following cancer treatment in patients with a low monocyte count and/or in patients having a Luminal B breast cancer and/or in patients with an age of less than about 85 years and/or in patients having been previously treated with a CDK4/6 inhibitor, and/or in patients having not previously undergone treatment with a taxane chemotherapy. Suitably, during maintenance therapy, the LAG-3 protein or derivative thereof is administered about every 1 week, or about every 2 weeks, or about every 3 weeks, or about every 4 weeks, for a period of up to about 48 weeks.

Further Patient Subgroups

In other embodiments, one or more further patient subgroups are selected for treatment. Such subgroups include, for example, patients who have a higher or lower initial performance status, patients who have previously received extensive exposure to corticosteroids, and patients with a low BMI e.g. <30 kg/m².

Pharmaceutical Compositions

The LAG-3 protein or derivative thereof and, where applicable, the chemotherapy agent are formulated with a pharmaceutically acceptable carrier, excipient, or diluent to provide a pharmaceutical composition. Typically these will be formulated as separate pharmaceutical compositions, although in the case of a fixed dose combination, the LAG-3 protein or derivative thereof and the chemotherapy agent will be formulated together, along with a pharmaceutically acceptable carrier, excipient, or diluent. The separate pharmaceutical compositions may be packaged together in the form of a kit-of-parts.

In general, the LAG-3 protein or derivative thereof and, where applicable, the chemotherapy agent may be administered by known means, in any suitable pharmaceutical composition, by any suitable route.

Suitable pharmaceutical compositions may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate compositions of the invention in a unit dosage form for ease of administration and uniformity of dosage. The term “unit dosage form” as used herein refers to physically discrete units suited as unitary dosages for the individuals to be treated. That is, the compositions are formulated into discrete dosage units each containing a predetermined “unit dosage” quantity of an active agent calculated to produce the desired therapeutic effect in association with the required pharmaceutical carrier, excipient or diluent. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

### Examples

Embodiments of the invention are now described, by way of example only, with reference to the accompanying drawings in which:

### Example 1—Active Immunotherapy Paclitaxel (AIPAC) in HER2⁻/HR⁺ Metastatic Breast Cancer (MBC)

A Phase IIb clinical study was carried out to investigate the safety and efficacy of the active immunotherapy IMP321 in combination (adjunctive) with paclitaxel chemotherapy in patients with HER2 negative hormone receptor-positive metastatic breast cancer.

A multicentre, placebo-controlled, double-blind, 1:1 randomised Phase IIb study in female HER2 negative hormone receptor-positive metastatic breast cancer patients was conducted. The study comprised two stages:
stage 1 which was an open-label, safety run-in stage consisting of cohorts 1 and 2 to confirm the recommended Phase II dose (RPTD) of IMP321 in combination with paclitaxel; and
stage 2 which was a placebo-controlled, double-blind randomisation stage, paclitaxel+IMP321 at the RPTD compared to paclitaxel+placebo.

Experimental:

Paclitaxel+IMP321 at the RPTD of 30 mg (114 patients):

The chemo-immunotherapy phase consisted of 6 cycles of 4 weeks. Patients received weekly paclitaxel at Days 1, 8 and 15 with adjunctive treatment of study agent (IMP321) on Days 2 and 16 of each 4-week cycle. After completion of the 6-cycle chemo-immunotherapy phase, responding or stable patients received study agent (IMP321) every 4 weeks during the maintenance phase for an additional period of up to 12 injections.

Active Comparator: Paclitaxel+Placebo (112 patients):

The chemo-immunotherapy phase consisted of 6 cycles of 4 weeks. Patients received weekly paclitaxel at Days 1, 8 and 15 with adjunctive treatment of study agent (placebo) on Days 2 and 16 of each 4-week cycle. After completion of the 6-cycle chemo-immunotherapy phase, responding or stable patients received study agent (placebo) every 4 weeks during the maintenance phase for an additional period of up to 12 injections.

Primary Outcome Measures:
1. Stage 1 to determine the recommended phase two dose (RPTD) for the randomised phase
2. Assessment of Progression-Free Survival (PFS)

Secondary Outcome Measures:
1. Assessment of the safety and tolerability of IMP321 compared to placebo
2. Assessment of the overall survival (OS)
3. Evaluation of pharmacokinetic parameters e.g. Peak Plasma Concentration [Cₘₐₓ]
4. Assessment of the change in quality of life (QOL)
5. Evaluation of the time to next treatment
6. Evaluation of objective response rate (ORR)
7. Evaluation of stable disease

Other Outcome Measures:
1. Assessment of immuno-monitoring in a defined subset of 60 patients during the randomised stage

TABLE 1
Baseline characteristics:

| | Paclitaxel + | Paclitaxel + |
|---|---|---|
| | IMP321 | Placebo |
| | (N = 114) | (N = 112) |
| Median age (range) | 58 yrs (24-87) | 61 yrs (35-79) |
| <65 years of age | 66.7% | 63.4% |
| ECOG 0 | 60.5% | 62.5% |
| % visceral disease | 90.4% | 92.9% |
| % pre-treated with CDK4/6 for met | 43.9% | 42.9% |
| disease | | |
| One or more systemic therapies for | 68.4% | 71.4% |
| metastatic disease | | |
| Tumor type (central pathology): | | |
| Luminal A | 34.1% | 36.7% |
| Luminal B | 48.8% | 49.4% |
| Monocytes at baseline <0.25 × 10⁹/L | 21.9% | 19.8% |

Well balanced treatment groups
Very late stage disease and large proportion pre-treated with CDK4/6

Results:

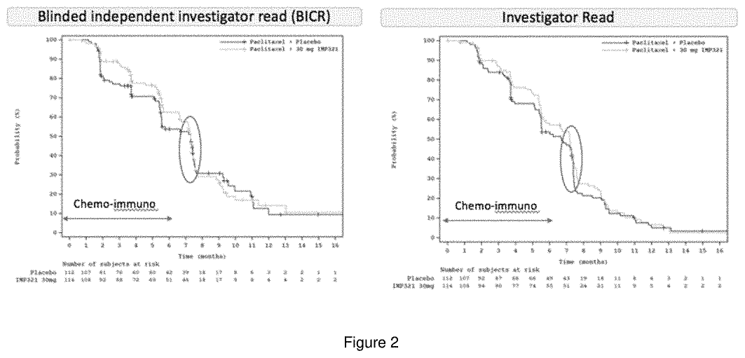

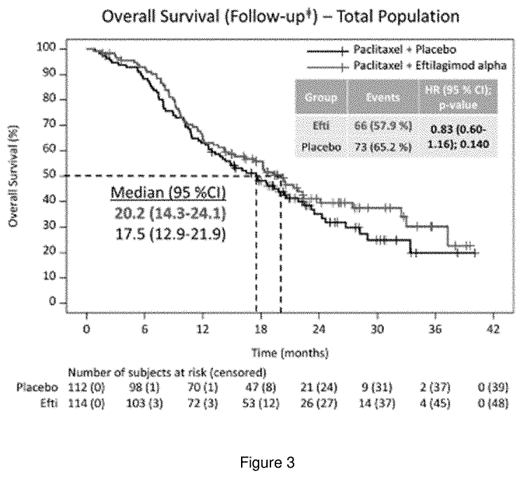

The results of the trial are shown in the tables below and accompanying figures. Results for the entire patient population are shown in Tables 2 to 4 below, and FIGS. 2 and 3.

TABLE 2
PFS improvement from paclitaxel + IMP321 vs. paclitaxel + placebo:

| | Paclitaxel + | Paclitaxel + | |
|---|---|---|---|
| | IMP321 | Placebo | Hazard Ratio |
| PFS estimates | (N = 114) | (N = 112) | [95% CI] |
| BICR: | | | |
| Median in months | 7.29 [6.64-7.46] | 7.29 [5.52-7.46] | 0.93 [0.67-1.30]¹ |
| [95% CI] | | | |
| Mean in months | 7.12 [0.37] | 6.64 [0.38] | |
| [SE] | | | |
| % progression | 63% [52%-71%] | 54% [43%-63%] | |
| free at 6 months | | | |
| Investigator read: | | | |
| Median in months | 7.16 [5.65-7.39] | 6.70 [5.52-7.33] | 0.92 [0.69-1.23]² |
| [95% CI] | | | |
| Mean in months | 6.81 [0.33] | 6.30 [0.31] | |
| [SE] | | | |
| % progression | 57% [47%-66%] | 54% [43%-63%] | |
| free at 6 months | | | |

¹p = 0.341
²p = 0.305

TABLE 3
OS improvement from paclitaxel + IMP321 vs. paclitaxel + placebo:

| | Paclitaxel + | Paclitaxel + | |
|---|---|---|---|
| | IMP321 | Placebo | Hazard Ratio |
| OS estimates | (N = 114) | (N = 112) | [95% CI] |
| Median in months | 20.2 [14.3-24.1] | 17.5 [12.9-21.9] | 0.83 [0.60-1.16]¹ |
| [95% CI] | | | |
| Investigator Read | | | |

¹p = 0.140

Table 3 and FIG. 3 show a positive trend for OS in the total population with a 2.7 month difference in the median OS in the respective groups.

TABLE 4
Efficacy improvement observed from paclitaxel + IMP321
compared to paclitaxel + placebo in terms of ORR:

| | Paclitaxel + | Paclitaxel + |
|---|---|---|
| | IMP321 | Placebo |
| BOR acc. to RECIST 1.1 by BICR | N = 114 | N = 112 |
| Complete Response | 0.9% | 1.8% |
| Partial Response | 47.4% | 36.6% |
| Stable disease | 36.8% | 37.5% |
| Progressive disease | 8.8% | 18.8% |
| Non-evaluable | 6.1% | 5.4% |
| Overall Response Rate (ORR)¹ | 48.3% | 38.4% |
| Disease Control Rate | 85.1% | 75.9% |

¹p = 0.118

Patient Subgroup with Low Monocytes at Baseline

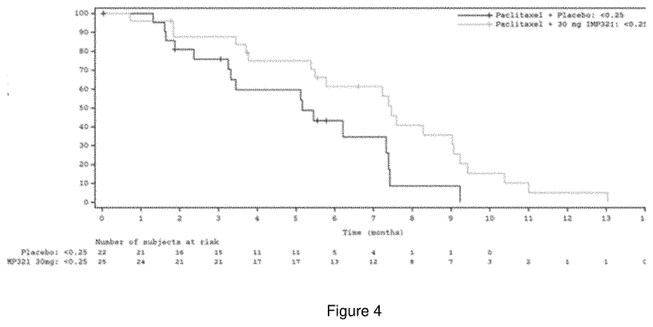

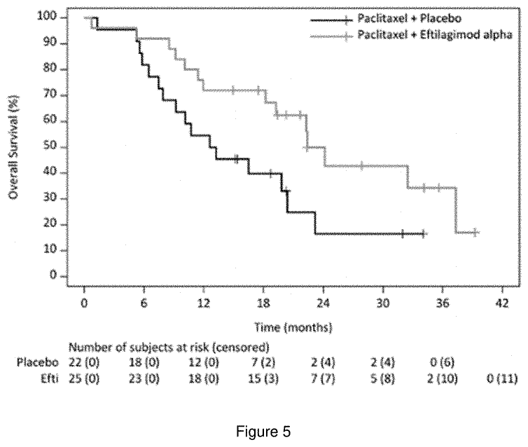

See Tables 5 to 7 below, and FIGS. 4 and 5.

TABLE 5
PFS improvement from paclitaxel + IMP321
vs. paclitaxel + placebo for patients
with low (<0.25 × 10⁹/L) monocytes at baseline:

| | Paclitaxel | Paclitaxel | | |
|---|---|---|---|---|
| PFS | + | + | | |
| estimates | IMP321 | Placebo | | |
| per | (Median, | (Median, | Hazard Ratio | Absolute |
| subgroup | months) | months) | [95% CI] | gain |
| BICR | 7.29 | 5.45 | 0.61 [0.29-1.15]¹ | +1.84 months |
| Investigator | 7.46 | 5.16 | 0.44 [0.21-0.90]² | +2.30 months |
| Read | | | | |

¹p = 0.084
²p = 0.012

TABLE 6
OS improvement from paclitaxel + IMP321 vs.
paclitaxel + placebo for patients
with low (<0.25 × 10⁹/L ) monocytes at baseline:

| | Paclitaxel + | Paclitaxel + | HR | Absolute |
|---|---|---|---|---|
| | IMP321 | Placebo | [95% CI] | gain |
| Median OS | 22.4 | 12.9 | 0.47 | +9.4 months |
| (Months) | [18.2-37.3] | [7.5-20.4] | [0.22-0.98]¹ | |
| [95% CI] | | | | |
| Investigator | | | | |
| Read | | | | |

¹p = 0.02

TABLE 7
Efficacy improvement observed from IMP321 compared
to placebo in terms of ORR and DCR
for low monocyte count (<0.25 × 10⁹/L) subgroup:

| | Paclitaxel + | | Paclitaxel + | |
|---|---|---|---|---|
| | IMP321 | | Placebo | |
| BORacc. to RECIST | <0.25 | >0.25 | <0.25 | >0.25 |
| 1.1 by BICR | N = 25 | N = 89 | N = 21 | N = 91 |
| Complete Response | 0.0% | 1.1% | 0.0% | 2.2% |
| Partial Response | 44.0% | 48.3% | 33.3% | 37.4% |
| Stable disease | 40.0% | 36.0% | 33.3% | 38.5% |
| Progressive disease | 12.0% | 7.9% | 28.6% | 16.5% |
| Non-evaluable | 4.0% | 6.7% | 4.8% | 5.5% |
| Overall Response Rate (ORR) | 44.0% | 49.4% | 33.3% | 39.6% |
| Disease Control Rate (DCR) | 84.0% | 85.4% | 66.7% | 78.1% |

Table 7 illustrates that the ORR and DCR for IMP321+paclitaxel is comparatively better than paclitaxel+placebo in the low monocyte count (<0.25×10⁹/L) subgroup. That is, in the low monocyte subgroup, patients treated with IMP321+paclitaxel had an ORR and DCR which was 10.7% and 17.3% better than patients treated with paclitaxel+placebo, respectively. By contrast, for patients with a baseline monocyte count of ≥0.25×10⁹/L, those treated with IMP321+paclitaxel had an ORR and DCR which was only 9.8% and 7.3% better than patients treated with paclitaxel+placebo, respectively.

FIGS. 4 (PFS) and 5 (OS) show the improvement of patients from this subgroup treated with paclitaxel+IMP321 versus paclitaxel+placebo.

Patient subgroup with Luminal B type

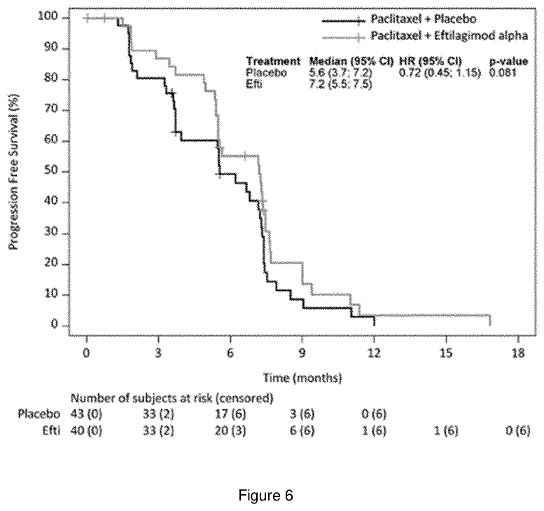

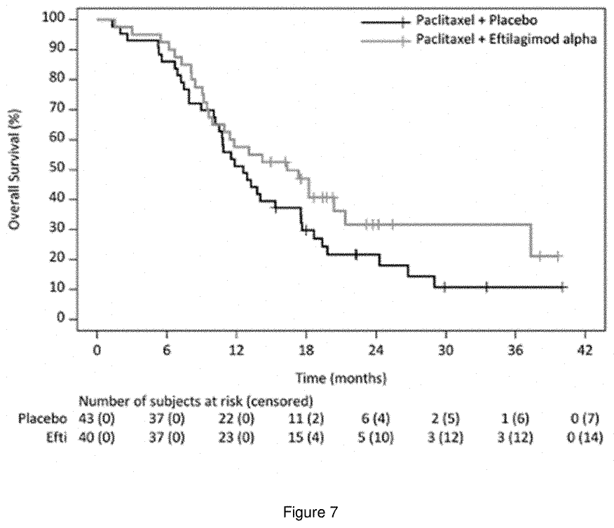

See Tables 8 to 10, and FIGS. 6 and 7.

TABLE 8
PFS improvement from paclitaxel + IMP321 vs.
paclitaxel + placebo for patients with Luminal B type:

| | Paclitaxel + | Paclitaxel + | | |
|---|---|---|---|---|
| PFS | IMP321 | Placebo | Hazard | |
| estimates per | (Median, | (Median, | Ratio | Absolute |
| subgroup | months) | months) | [95% CI] | gain |
| BICR | 7.29 | 5.45 | 0.65 | +1.84 months |
| Investigator | | | [0.38-1.11]¹ | |
| Read | 7.20 | 5.55 | 0.72 | +1.65 months |
| | | | [0.45-1.15]² | |

¹p = 0.058
²p = 0.081

TABLE 9
OS improvement from paclitaxel + IMP321 vs.
paclitaxel + placebo for patients with Luminal B type:

| | Paclitaxel + | Paclitaxel + | | |
|---|---|---|---|---|
| | IMP321 | Placebo | | |
| | (Median, | (Median, | HR | Absolute |
| | months) | months) | [95% CI] | gain |
| Median OS | 16.3 | 12.6 | 0.69 | +3.8 months |
| Investigator | [9.9-21.4] | [10.2-17.5] | [0.42-1.15]¹ | |
| Read | | | | |

¹p = 0.077

TABLE 10
Efficacy improvement observed from IMP321 compared
to placebo in terms of ORR and DCR for Luminal B subgroup:

| BOR acc. | Paclitaxel + IMP321 | | Paclitaxel + Placebo | |
|---|---|---|---|---|
| to RECIST | Luminal A | Luminal B | Luminal A | Luminal B |
| 1.1 by BICR | N = 31 | N = 40 | N = 36 | N = 43 |
| Complete Response | 3.23% | 0.00% | 2.78% | 2.33% |
| Partial Response | 48.39% | 42.50% | 41.67% | 30.23% |
| Stable disease | 38.71% | 40.00% | 33.33% | 39.53% |
| Progressive disease | 9.68% | 10.00% | 19.44% | 20.93% |
| Non-evaluable | 0.00% | 7.50% | 2.78% | 6.98% |
| Overall Response | 51.61% | 42.50% | 44.45% | 32.56% |
| Rate (ORR) | | | | |
| Disease Control | 90.32% | 82.50% | 77.78% | 72.09% |
| Rate (DCR) | | | | |

Table 10 illustrates that the ORR for IMP321+paclitaxel is comparatively better than paclitaxel+placebo in Luminal B patients. That is, Luminal B patients treated with IMP321+paclitaxel had an ORR which was 9.9% better than patients treated with paclitaxel+placebo. By contrast, for Luminal A patients, those treated with IMP321+paclitaxel had an ORR which was 7.2% better than patients treated with paclitaxel+placebo, respectively.

FIGS. 6 (PFS) and 7 (OS) show the improvement of patients from this subgroup treated with paclitaxel+IMP321 versus paclitaxel+placebo.

Age Based Patient Subgroup

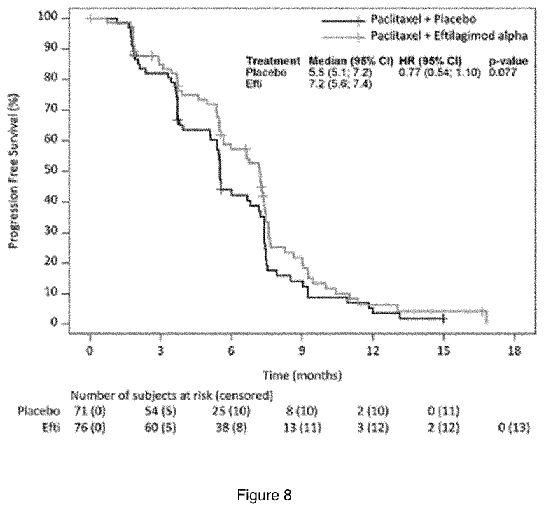

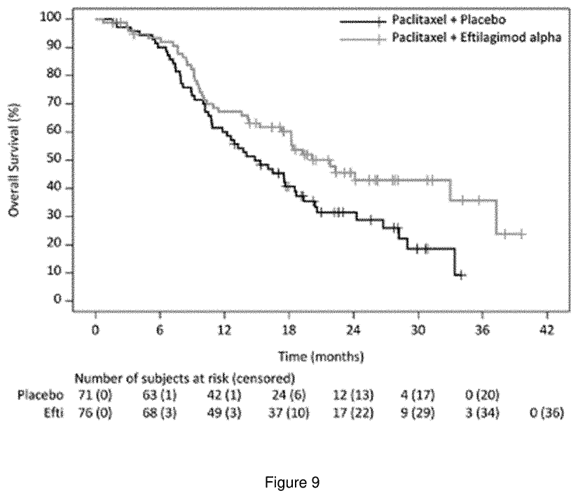

See Tables 11 and 12, and FIGS. 8 and 9.

TABLE 11
PFS improvement from paclitaxel + IMP321 vs.
paclitaxel + placebo for patients with age < 65 years:

| | Paclitaxel + | Paclitaxel + | | |
|---|---|---|---|---|
| | IMP321 | Placebo | | |
| | (Median, | (Median, | HR | Absolute |
| | months) | months) | [95% CI] | gain |
| Median PFS | 7.2 | 5.5 | 0.77 | +1.7 months |
| Investigator | [5.6-7.4] | [5.1-7.2] | [0.54-1.10]¹ | |
| read | | | | |

¹p = 0.077

TABLE 12
OS improvement from paclitaxel + IMP321 vs.
paclitaxel + placebo for patients with age < 65 years:

| | Paclitaxel + | Paclitaxel + | | |
|---|---|---|---|---|
| | IMP321 | Placebo | | |
| | (Median, | (Median, | HR | Absolute |
| | months) | months) | [95 CI] | gain |
| Median OS | 21.9 | 14.8 | 0.62 | +7.1 months |
| Investigator | [15.3-37.3] | [10.9-18.7] | [0.41-0.94]¹ | |
| read | | | | |

¹p = 0.012

FIGS. 8 (PFS) and 9 (OS) show the improvement of patients from this subgroup treated with paclitaxel+IMP321 versus paclitaxel+placebo.

Patient Subgroup with Prior Treatment with a CDK4/6 Inhibitor

TABLE 13
PFS from paclitaxel + IMP321 vs. paclitaxel +
placebo for patients with prior CDK4/6 therapy:

| | Paclitaxel + | Paclitaxel + | | |
|---|---|---|---|---|
| | IMP321 | Placebo | Hazard | |
| PFS estimates | (Median, | (Median, | Ratio | Absolute |
| per subgroup | months) | months) | [95% CI] | gain |
| Median PFS | 7.13 | 5.55 | 0.99 | +1.58 |
| (With prior | | | [0.61-1.60]¹ | months |
| CDK4/6 therapy) | 5.55 | 6.70 | 1.02 | −1.15 |
| BICR | | | [0.67-1.57]² | months |
| Investigator Read | | | | |

¹p = 0.489
²p = 0.556

TABLE 14
OS improvement from paclitaxel + IMP321 vs. paclitaxel +
placebo for patients with prior (and without prior) CDK4/6 therapy:

| | Paclitaxel + | Paclitaxel + | | |
|---|---|---|---|---|
| | IMP321 | Placebo | | |
| | (Median, | (Median, | Hazard | Absolute |
| | months) | months) | Ratio | gain |
| Median OS | 20.2 | 14.9 | 0.84 | +5.3 months |
| (With prior | | | | |
| CDK4/6 therapy) | | | | |
| Investigator Read | | | | |
| Median OS | 20.4 | 19.8 | 0.84 | +0.6 months |
| (Without prior | | | | |
| CDK4/6 therapy) | | | | |
| Investigator Read | | | | |

Patient Subgroup without Prior Taxane Therapy

TABLE 15
PFS from paclitaxel + IMP321 vs. paclitaxel +
placebo for patients without prior taxane therapy:

| | Paclitaxel + | Paclitaxel + | | |
|---|---|---|---|---|
| | IMP321 | Placebo | Hazard | |
| PFS estimates | (Median, | (Median, | Ratio | Absolute |
| per subgroup | months) | months) | [95% CI] | gain |
| Median PFS | 7.39 | 7.29 | 0.85 | +0.10 |
| (Without prior | | | [0.55-1.32]¹ | months |
| taxane therapy) | 7.16 | 5.52 | 0.88 | +1.64 |
| BICR | | | [0.58-1.25]² | months |
| Investigator Read | | | | |

¹p = 0.232
²p = 0.216

TABLE 16
OS improvement from paclitaxel + IMP321 vs. paclitaxel +
placebo for patients without prior taxane therapy:

| | Paclitaxel + | Paclitaxel + | | |
|---|---|---|---|---|
| | IMP321 | Placebo | | |
| | (Median, | (Median, | Hazard | Absolute |
| | months) | months) | Ratio | gain |
| Median OS | 21.36 | 18.53 | 0.69¹ | +2.83 months |
| (Without prior | | | | |
| taxane therapy) | | | | |
| Investigator Read | | | | |

¹p = 0.056

TABLE 17
Summary of subgroup data (Low Monocytes, Luminal B, Age, with prior CDK4/6
Therapy and without prior taxane therapy) by investigator read from patients treated
with paclitaxel + IMP321 versus paclitaxel + placebo:

| | | | Absolute | Median | Absolute |
|---|---|---|---|---|---|
| Subgroup | Treatment | Median PFS | PFS Gain | OS | OS Gain |
| Low | IMP321 | 7.5 | +2.3 months | 22.4 | +9.4 months |
| monocytes | Placebo | 5.2 | HR 0.44 | 12.9 | HR 0.47 |
| | | | p = 0.012 | | p = 0.02 |
| Luminal B | IMP321 | 7.2 | +1.7 months | 16.3 | +3.8 months |
| | Placebo | 5.6 | HR 0.72 | 12.6 | HR 0.69 |
| | | | p = 0.081 | | p = 0.077 |
| <65 yrs | IMP321 | 7.2 | +1.7 months | 21.9 | +7.1 months |
| | Placebo | 5.5 | HR 0.77 | 14.8 | HR 0.62 |
| | | | p = 0.077 | | p = 0.012 |
| With prior | IMP321 | 5.6 | −1.1 months | 20.2 | +5.3 months |
| CDK4/6 | Placebo | 6.7 | HR 1.02 | 14.9 | HR 0.84 |
| therapy | | | p = 0.556 | | |
| Without | IMP321 | 7.2 | +1.7 months | 21.4 | +2.8 months |
| prior taxane | Placebo | 5.5 | HR 0.88 | 18.5 | HR 0.69 |
| therapy | | | p = 0.216 | | p = 0.056 |

Primary analysis used for safety and PFS cut-off: Jan. 9th 2020;
Follow-up 2 used for OS cut off: 24th Sep. 2020 (~60% events)

Patient Subgroup with Higher or Lower ECOG Performance Status at Baseline

To conduct clinical trials for the treatment of cancer in a consistent manner across many participating hospitals, cancer centres, and clinics requires the use of standard criteria for measuring how the disease impacts a patient's daily living abilities (i.e. a patient's performance status). The ECOG Scale of Performance Status is one such measurement. It describes a patient's level of functioning in terms of their ability to care for themselves, daily activity, and physical ability (walking, working, etc.).

Researchers worldwide take the ECOG Performance Status into consideration when planning trials to study a new treatment method. This numbering scale is one way to define the population of patients to be studied in the trial, so that it can be uniformly reproduced among physicians who enrol patients. It is also a way for physicians to track changes in a patient's level of functioning as a result of treatment during the trial.

The scale was developed by the Eastern Cooperative Oncology Group (ECOG), now part of the ECOG-ACRIN Cancer Research Group, and published in 1982 (Oken et al., “Toxicity and response criteria of the Eastern Cooperative Oncology Group”. Am J Clin Oncol. 1982; 5:649-655).

TABLE 18
ECOG Performance Status:

| Grade | ECOG Performance Status |
|---|---|
| 0 | Fully active, able to carry on all pre- |
| | disease performance without restriction |
| 1 | Restricted in physically strenuous |
| | activity but ambulatory and able to |
| | carry out work of a light or sedentary |
| | nature, e.g., light house work, office |
| | work |
| 2 | Ambulatory and capable of all selfcare |
| | but unable to carry out any work |
| | activities; up and about more than 50% |
| | of waking hours |
| 3 | Capable of only limited selfcare; |
| | confined to bed or chair more than 50% |
| | of waking hours |
| 4 | Completely disabled; cannot carry on |
| | any selfcare; totally confined to bed or |
| | chair |
| 5 | Dead |

TABLE 19
Progression Free Survival (PFS) improvement from
paclitaxel + IMP321 vs. paclitaxel + placebo for
patients with lower performance status (>0) at baseline:

| | Paclitaxel + | Paclitaxel + | Hazard Ratio |
|---|---|---|---|
| PFS estimates | IMP321 | Placebo | [95 CI]; |
| per subgroup | (Median, months) | (Median, months) | p-value |
| Median PFS | 7.13 | 6.67 | 0.76 |
| ECOG > 0 | 6.64 | 5.52 | [0.43-1.35]¹ |
| BICR | | | 0.67 |
| Investigator | | | [0.42-1.09]² |
| Read | | | |
| Median PFS | 7.29 | 7.43 | 1.06 |
| ECOG = 0 | 7.23 | 7.23 | [0.74-1.53]³ |
| BICR | | | 1.01 |
| Investigator | | | [0.66-1.55]⁴ |
| Read | | | |

¹p = 0.178
²p = 0.057
³p = 0.634
⁴p = 0.526

TABLE 20
OS from paclitaxel + IMP321 vs. paclitaxel + placebo
in patients with differing ECOG performance status (0 or >0):

| | Paclitaxel + | Paclitaxel + | | |
|---|---|---|---|---|
| | IMP321 | Placebo | | |
| | (Median, | (Median, | | Absolute |
| | months) | months) | HR | gain |
| Median OS | 22.37 (0) | 17.68 (0) | 0.80 | +4.69 months |
| Investigator | 14.22 (>0) | 12.83 (>0) | 0.88 | +1.39 months |
| Read | | | | |

Interestingly, in contrast to the PFS data, the OS data showed a more meaningful improvement from treatment with paclitaxel+IMP321 compared with paclitaxel+placebo in patients having an ECOG performance status of 0, possibly reflective of their better health including the health of their immune system (and the ability to meaninfully respond to therapy with an active immunotherapy), compared with those patients having an ECOG performance status of >0. As explained in the multivariate analysis below, ECOG status is likely a prognostic factor, rather than a predictive factor.

Overall, the subgroup data generally fit well with the mechanism of action of IMP321.

Immune Monitoring

Immune monitoring studies were undertaken. Blood samples from selected patients in the clinical trial were collected prior to paclitaxel administration (i.e. 13 days after the 6^{th}/12^{th} injection of placebo or IMP321) to monitor the absolute counts of T cells by flow cytometry.

The mean number of T cells+/−SEM in patients from the placebo (n=36) and IMP321 (n=31) groups is presented at each timepoint. The difference between the two groups was tested by Wilcoxon rank sum tests.

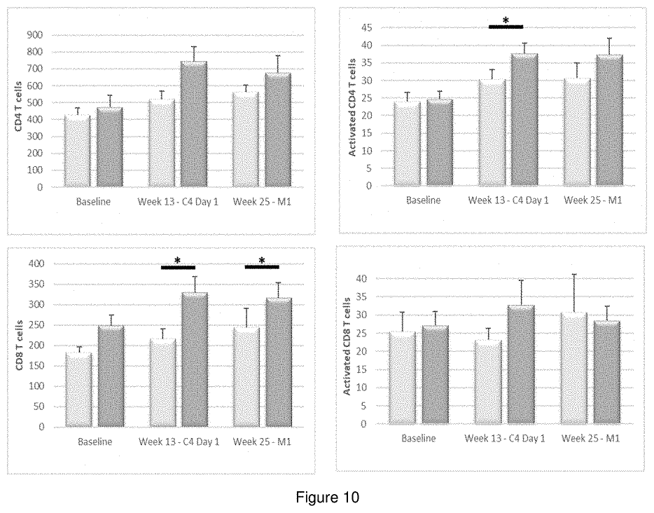

FIG. 10 shows that patients treated with paclitaxel+IMP321 had increased amounts of circulating CD4 and CD8 T cells compared with patients treated with paclitaxel+placebo.

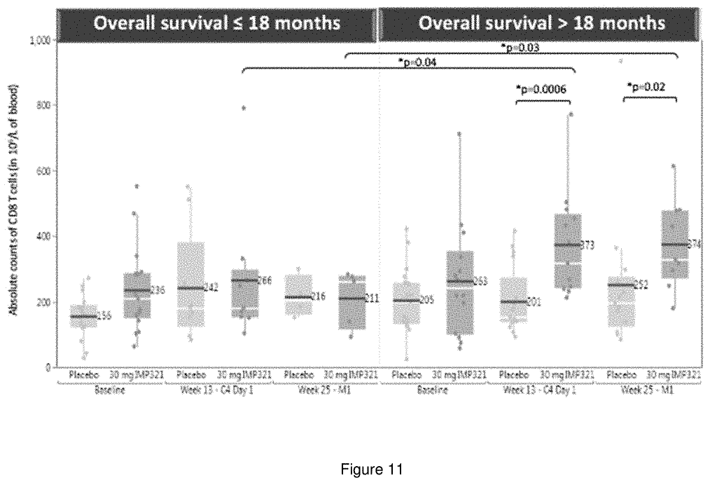

Importantly, FIG. 11 illustrates that the increase in the number of circulating CD8 T cells in patients treated with paclitaxel+IMP321 is correlated with those patients who have a long term benefit (OS>18 months) from the chemo-immunotherapy treatment.

Multivariate Analysis

Multivariate analysis was undertaken using the Cox model to identify (a) prognostic factors that may serve for stratification in a Phase III trial and (b) predictive factors that show in which patient populations the largest treatment effect may be seen. The following factors were used in the cox model:
Age category (<65 years/>=65 years)
Time diagnosis to IC (metric)
ECOG performance status (0/>0)—stratification factor
BMI at baseline (<25/>=25)
Liver metastasis (Y/N)
Prior CDK4/6 treatment (Y/N)
Prior taxane treatment (Y/N)
Use of systemic corticosetroids (Limited/Extended)
Lympocytes at baseline (<0.75/0.75−1.30/>=1.30)
Monocytes at baseline (<0.25/>=0.25)
LDH at baseline (<=250/>250)
Number of disease sites (<=2/>2)

In the final model, ECOG status and prior CDK4/6 treatment were identified as prognostic factors for stratification in a future clinical trial.

Low monocyte count, age and no prior taxane therapy were identified as the more important predictive factors, as follows:

TABLE 21
shows the results of the multivariate
model for PFS (investigator read):

| Subgroup | Interaction p |
|---|---|
| Age category | 0.0997 |
| ECOG (stratification factor) | 0.0499 |
| BMI | 0.7710 |
| Liver metastasis | 0.5154 |
| Prior CDK4/6 treatment | 0.6997 |
| Prior taxane treatment | 0.7503 |
| Use of systemic corticosetroids | n.e. |
| Lympocytes at baseline | 0.9091 |
| Monocytes at baseline | 0.0526 |
| LDH at baseline | 0.9232 |
| Number of disease sites | 0.2575 |

TABLE 22
shows the results of the multivariate
model for OS (investigator read):

| Subgroup | Interaction p |
|---|---|
| Age category | 0.1131 |
| ECOG | 0.5252 |
| BMI | 0.9224 |
| Liver metastasis | 0.6154 |
| Prior CDK4/6 treatment | 0.9922 |
| Prior taxane treatment | 0.0402 |
| Use of systemic corticosteroids | n.e. |
| Lympocytes at baseline | 0.7596 |
| Monocytes at baseline | 0.0393 |
| LDH at baseline | 0.1421 |
| Number of disease sites | 0.5355 |

Conclusions:
In the entire patient population, the IMP321 group had a higher response rate and fewer patients with immediate progression compared with the placebo group
Progression Free Survival (PFS) Hazard Ratio improvement for IMP321 group versus placebo group after 6 months
Increased Overall Response Rate (ORR) of 48.3% in the IMP321 group versus 38.4% in the placebo group
Significant deterioration in Quality of Life (QoL) in the placebo group at week 25, which was not observed in the IMP321 group
Combination therapy was safe and well tolerated

63% of patients who received paclitaxel plus IMP321 were progression-free after 6 months (at the end of the chemo-immunotherapy combination phase) and according to RECIST 1.1 based on blinded independent central readers (BICR). This compares favourably to 54% of patients who received paclitaxel plus placebo. The PFS data yielded an unadjusted hazard ratio (HR) of 0.93. The secondary endpoint of Overall Response Rate (ORR) increased to 48.3% in the IMP321 group, from 38.4% in the placebo group. In terms of OS, there was a favourable and improving trend favouring the IMP321 treatment group, with a gain of 2.7 months in median OS and a HR of 0.83.

Consistent with embodiments of the present invention, particularly favourable and unexpected results (compared with standard of care chemotherapy) were reported in multiple predefined patient subgroups as illustrated in Tables 5 to 17, 21 and 22, and FIGS. 4 to 9:
patients with low monocyte count at baseline had a positive HR of 0.44 (median PFS of 5.2 vs. 7.5 months) and 0.47 (median OS of 12.9 vs. 22.4 months) favouring IMP321;
patients with a more aggressive, more immunogenic Luminal B type MBC had a positive HR of 0.72 (median PFS of 5.6 vs. 7.2 months) and 0.69 (median OS of 12.6 vs. 16.3 months) favouring IMP321;
patients with an age of <65 years had a positive HR of 0.77 (median PFS of 5.5 vs. 7.2 months) and 0.62 (median OS of 14.8 vs. 21.9 months) favouring IMP321;
for those patients pre-treated with a CDK4/6 inhibitor, they had a positive OS gain of 5.3 months when treated with IMP321 compared with placebo. This is important as treatment with CDK4/6 inhibtors has, in recent years, rapidly become the standard of care for 1St line treatment of HR+/HER2− metastatic breast cancer;
patients not having undergone prior taxane chemotherapy had a positive OS gain of 2.8 months from treatment with IMP321; and
multivariate analysis indicated that low monocyte count, age (e.g. <65 years) and no prior taxane therapy were identified as important predictive factors where the largest treatment effect can be seen.

A summary of the data for the key subgroups (Low Monocytes, Luminal B, Age, with prior CDK4/6 Therapy, and without prior taxane therapy) is provided in Table 17 showing meaningful absolute gains in PFS and/or OS in accordance with the present invention. These meaningful gains are surprising and unexpected as there has been no improvement in recent years in terms of treatment options for HR+/HER2− metastatic breast cancer patients eligible to receive chemotherapy (i.e. following endocrine therapy with/without treatment with a CDK4/6 inhibitor). Furthermore, there are no active immunotherapies currently approved or in late-stage trials for this indication.

1. A LAG-3 protein, or a derivative thereof that is able to bind to MHC class II molecules, for use in preventing, treating, or ameliorating a cancer in a subject with one or more of a low monocyte count, a Luminal B breast cancer, an age of less than about 85 years, has been previously treated with a CDK4/6 inhibitor, and has not previously undergone treatment with a taxane chemotherapy.

2. Use of a LAG-3 protein, or a derivative thereof that is able to bind to MHC class II molecules, in the manufacture of a medicament for the prevention, treatment, or amelioration of a cancer in a subject with one or more of a low monocyte count, a Luminal B breast cancer, an age of less than about 85 years, has been previously treated with a CDK4/6 inhibitor, and has not previously undergone treatment with a taxane chemotherapy.

3. A LAG-3 protein, or a derivative thereof, for use according to claim 1, or use according to claim 2, wherein the cancer is a breast cancer.

4. A LAG-3 protein, or a derivative thereof, for use, or use, according to any preceeding claim, wherein the cancer is a hormone receptor-positive breast cancer.

5. A LAG-3 protein, or a derivative thereof, for use, or use, according to any preceeding claim, wherein the cancer is a hormone receptor-positive HER2 negative breast cancer.

6. A LAG-3 protein, or a derivative thereof, for use, or use, according to any preceeding claim, wherein the cancer is metastatic breast cancer.

7. A LAG-3 protein, or a derivative thereof, for use, or use, according to any preceding claim, wherein the LAG-3 protein, or derivative thereof, is to be administered before, with, or after administration of a chemotherapy agent.

8. A LAG-3 protein, or a derivative thereof, for use, or use, according to claim 7, wherein the LAG-3 protein, or derivative thereof, is to be administered after administration of the chemotherapy agent.

9. A LAG-3 protein, or a derivative thereof, for use, or use, according to claim 7 or 8, wherein the chemotherapy agent is a taxane.

10. A LAG-3 protein, or a derivative thereof, for use, or use, according to claim 9, wherein the taxane is paclitaxel.

11. A LAG-3 protein, or a derivative thereof, for use, or use, according to any preceeding claim, wherein the subject has one or more of a low monocyte count, an age of less than about 85 years, and has not previously undergone treatment with a taxane chemotherapy.

12. A LAG-3 protein, or a derivative thereof, for use, or use, according to any preceeding claim, wherein the subject has been previously treated with a CDK4/6 inhibitor and has one or more of a low monocyte count, an age of less than about 85 years, and has not previously undergone treatment with a taxane chemotherapy.

13. A LAG-3 protein, or a derivative thereof, for use, or use, according to any preceeding claim, wherein the subject has an age of less than about 85 years and the subject has not previously undergone treatment with a taxane chemotherapy.

14. A LAG-3 protein, or a derivative thereof, for use, or use, according to any of claims 1 to 12, wherein the subject has an age of less than about 85 years.

15. A LAG-3 protein, or a derivative thereof, for use, or use, according to any preceeding claim, wherein the subject has an age of less than about 65 years.

16. A LAG-3 protein, or a derivative thereof, for use, or use, according to any preceeding claim, wherein the derivative of LAG-3 comprises:
the 30 amino acid extra-loop sequence GPPAAAPGHPLAPGPHPAAPSSWGPRPRRY (SEQ ID NO:2) of domain D1 of human LAG-3 protein; or
a variant of the 30 amino acid extra-loop sequence GPPAAAPGHPLAPGPHPAAPSSWGPRPRRY (SEQ ID NO:2) of domain D1 of human LAG-3 protein, wherein the variant comprises one or more conservative amino acid substitutions, and has at least 70% amino acid identity with the 30 amino acid extra-loop sequence.

17. A LAG-3 protein, or a derivative thereof, for use, or use, according to any preceeding claim, wherein the derivative of LAG-3 protein comprises an amino acid sequence that has at least 70% amino acid identity with domain D1, and optionally domain D2, of LAG-3 protein, or at least 70% amino acid identity with domains D1, D2, D3, and optionally D4, of LAG-3 protein.

18. A LAG-3 protein, or a derivative thereof, for use, or use, according to any preceeding claim, wherein the derivative of LAG-3 protein is fused to Immunoglobulin Fc sequence.

19. A LAG-3 protein, or a derivative thereof, for use, or use, according to any preceeding claim, wherein the derivative of LAG-3 protein is IMP321.

20. A LAG-3 protein, or a derivative thereof, for use, or use, according to any preceeding claim, wherein the LAG-3 protein, or derivative thereof, is present at a dose which is a molar equivalent of about 6 mg to about 60 mg, about 10 mg to about 50 mg, about 20 mg to about 40 mg, about 25 mg to about 35 mg, or about 30 mg of the LAG-3 derivative LAG-31g fusion protein IMP321.


SEQUENCE LISTING (2 sequences)

SEQ ID NO: 1 - protein, 502 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPQAQSKIEELEQEPEPEPEPEPEPEPEPEPEQL

SEQ ID NO: 2 - protein, 30 aa, Homo sapiens
GPPAAAPGHPLAPGPHPAAPSSWGPRPRRY